(19) United States
(12) Patent Application Publication
(10) Pub. No.: US 2026/0065556 A1
(43) Pub. Date: Mar. 5, 2026

(54) INFORMATION PROCESSING APPARATUS, INFORMATION PROCESSING METHOD, AND INFORMATION PROCESSING PROGRAM

(71) Applicant: FUJIFILM Corporation, Tokyo (JP)

(72) Inventor: Aya IMAMURA, Tokyo (JP)

(73) Assignee: FUJIFILM Corporation, Tokyo (JP)

(21) Appl. No.: 19/306,999

(22) Filed: Aug. 21, 2025

(30) Foreign Application Priority Data
Aug. 29, 2024 (JP) 2024-147472

(51) Int. Cl. G06T 11/00; A61B 6/00; A61B 6/03; A61B 6/50

(52) U.S. Cl. CPC G06T 12/00; A61B 6/032; A61B 6/504; A61B 6/5211; G06T 2210/41; G06T 2211/40

(57) ABSTRACT

A processor is configured to acquire a plurality of CT images derived by reconstructing a plurality of pieces of projection data having different energy levels, which are acquired by imaging a subject at a plurality of timings, at a plurality of predetermined energy levels, derive visibility information related to visibility of a target region included in each of the plurality of CT images, and determine an image interpretation energy level for interpreting the CT image among the plurality of energy levels based on the visibility information.

### CROSS REFERENCE TO RELATED APPLICATIONS

The present application claims priority from Japanese Patent Application No. 2024-147472, filed on Aug. 29, 2024, the entire disclosure of which is incorporated herein by reference.

### BACKGROUND

### Technical Field

The present disclosure relates to an information processing apparatus, an information processing method, and an information processing program.

### Related Art

In a dynamic contrast examination of the liver performed using a computed tomography (CT) apparatus or a magnetic resonance imaging (MRI) apparatus, a plurality of CT images (dynamic contrast CT images) having different contrast phases are acquired at a plurality of timings while a contrast agent is injected, and a change in degree of enhancement of a lesion portion over time is observed.

On the other hand, the dual-energy (DE) CT device and the photon counting (PC) CT device acquire the energy information of the X-rays. Such a DECT device and a PCCT device comprise a technology of spectral imaging that performs image reconstruction by performing material decomposition. For example, in the DECT device, it is possible to generate a virtual monochromatic X-ray image such as the image captured by X-rays at any single energy level by reconstructing two pieces of projection data captured at two types of tube voltages at any single energy level. In addition, in the PCCT device, it is possible to generate a material decomposition image that decomposes and visualizes a plurality of substances having different attenuation coefficients of X-rays or to generate a virtual monochromatic X-ray image by reconstructing a plurality of pieces of projection data having different energy levels acquired for each energy bin at any single energy level. In addition, even in the DECT device, it is possible to generate the material decomposition image using the projection data captured at different tube voltages.

Here, in the dynamic contrast CT image, the visibility of the lesion varies for each combination of the contrast phase and the energy level. In addition, the optimal energy level for the image interpretation also varies depending on the body type of the patient and the like. Therefore, it is ideal to generate virtual monochromatic X-ray images having various energy levels and the like in each of the plurality of contrast phases such that the radiologist can comprehensively check the images, which improves the diagnostic accuracy.

However, in a case where virtual monochromatic X-ray images having various energy levels or the like are generated in each of the plurality of contrast phases, the size of the image is enormous. In addition, since the number of images to be interpreted by the doctor is also enormous, the burden on the doctor is large.

Therefore, for example, a method has been proposed in which a map image including spatial information about a region of interest and information representing a change in an analysis target value associated with a change in effective X-ray energy is generated to reduce the burden on the doctor (see JP2019-072082A).

However, in the method described in JP2019-072082A, the change over time, which is important for the discrimination of the lesion, is not considered.

### SUMMARY OF THE INVENTION

The present disclosure has been made in view of the above-described circumstances, and an object of the present disclosure is to make it easy to see a change over time in a CT image in a case where the CT image is acquired at a plurality of timings.

An information processing apparatus according to the present disclosure comprises
a processor,
in which the processor is configured to:
   acquire a plurality of CT images derived by reconstructing a plurality of pieces of projection data having different energy levels, which are acquired by imaging a subject at a plurality of timings, at a plurality of predetermined energy levels;
   derive visibility information related to visibility of a target region included in each of the plurality of CT images; and
   determine an image interpretation energy level for interpreting the CT image among the plurality of energy levels based on the visibility information.

In the information processing apparatus according to the present disclosure, the processor may be configured to:
derive an amount of change representing a change in the visibility of the target region over time at each of the plurality of energy levels, as the visibility information; and
determine the image interpretation energy level by comparing the visibility information for each of the plurality of energy levels.

In the information processing apparatus according to the present disclosure, the processor may be configured to determine an energy level at which the amount of change is maximum among the plurality of energy levels, as the image interpretation energy level.

In the information processing apparatus according to the present disclosure, the processor may be configured to:
derive a representative value representing the visibility of the target region at each of the plurality of timings, as the visibility information; and
determine the image interpretation energy level by comparing the representative value for each of the plurality of timings.

In the information processing apparatus according to the present disclosure, the processor may be configured to determine the image interpretation energy level based on an energy level at which the representative value at each of the plurality of timings is optimal.

In the information processing apparatus according to the present disclosure, the processor may be configured to:
derive an amount of change in the visibility of the target region over time at each of the plurality of energy levels and a representative value representing the visibility of the target region at each of the plurality of timings, as the visibility information; and
determine the image interpretation energy level based on the amount of change and the representative value.

In the information processing apparatus according to the present disclosure, the processor may be configured to:
weight at least one of the amount of change or the representative value in accordance with the amount of change and the representative value; and
determine the image interpretation energy level based on the weighted amount of change and representative value.

In the information processing apparatus according to the present disclosure, the processor may be configured to sequentially display the plurality of CT images at the image interpretation energy level in chronological order.

In the information processing apparatus according to the present disclosure, the processor may be configured to further display an image of the target region included in the CT image having a highest visibility in association with the plurality of CT images at the image interpretation energy levels at each of the plurality of timings.

In the information processing apparatus according to the present disclosure, the processor may be configured to switch between display and non-display of the image of the target region in accordance with an instruction from an operator.

In the information processing apparatus according to the present disclosure, the CT image may be a virtual monochromatic X-ray image derived by reconstructing the plurality of pieces of projection data having different energy levels at the plurality of predetermined energy levels.

In the information processing apparatus according to the present disclosure, the CT image may be a material decomposition image derived by reconstructing the plurality of pieces of projection data having different energy levels at the plurality of predetermined energy levels.

In the information processing apparatus according to the present disclosure, the processor may be configured to derive the visibility information based on a feature value of a region including the target region included in the CT image.

In the information processing apparatus according to the present disclosure, the plurality of timings may be based on a contrast phase in a case where a contrast agent is injected into the subject.

In the information processing apparatus according to the present disclosure, the target region may be a lesion region.

An information processing method according to the present disclosure comprises
causing a computer to
   acquire a plurality of CT images derived by reconstructing a plurality of pieces of projection data having different energy levels, which are acquired by imaging a subject at a plurality of timings, at a plurality of predetermined energy levels,
   derive visibility information related to visibility of a target region included in each of the plurality of CT images, and
   determine an image interpretation energy level for interpreting the CT image among the plurality of energy levels based on the visibility information.

An information processing program according to the present disclosure causes a computer to execute:
a procedure of acquiring a plurality of CT images derived by reconstructing a plurality of pieces of projection data having different energy levels, which are acquired by imaging a subject at a plurality of timings, at a plurality of predetermined energy levels;
a procedure of deriving visibility information related to visibility of a target region included in each of the plurality of CT images; and
a procedure of determining an image interpretation energy level for interpreting the CT image among the plurality of energy levels based on the visibility information.

The technology of the present disclosure may be applied to a program product.

According to the present disclosure, it is possible to make it easy to see a change over time in a CT image in a case where the CT image is acquired at a plurality of timings.

### BRIEF DESCRIPTION OF THE DRAWINGS

FIG. 1 is a diagram showing a schematic configuration of a medical information system to which an information processing apparatus according to a first embodiment of the present disclosure is applied.

FIG. 2 is a diagram showing virtual monochromatic X-ray images of an arterial phase, a portal venous phase, and an equilibrium phase at a plurality of energy levels.

FIG. 3 is a diagram showing a hardware configuration of the information processing apparatus according to the first embodiment.

FIG. 4 is a functional configuration diagram of the information processing apparatus according to the first embodiment.

FIG. 5 is a diagram showing extraction of a lesion region.

FIG. 6 is a diagram showing feature values of a lesion region included in virtual monochromatic X-ray images of the arterial phase, the portal venous phase, and the equilibrium phase at the plurality of energy levels in the first embodiment.

FIG. 7 is a diagram showing a display screen of the virtual monochromatic X-ray image in the first embodiment.

FIG. 8 is a flowchart showing processing performed in the first embodiment.

FIG. 9 is a diagram showing feature values of a lesion region included in virtual monochromatic X-ray images of an arterial phase, a portal venous phase, and an equilibrium phase at a plurality of energy levels in a second embodiment.

FIG. 10 is a flowchart showing processing performed in the second embodiment.

FIG. 11 is a diagram showing feature values of a lesion region included in virtual monochromatic X-ray images of an arterial phase, a portal venous phase, and an equilibrium phase at a plurality of energy levels in a third embodiment.

FIG. 12 is a diagram showing a display screen of a virtual monochromatic X-ray image in the third embodiment.

FIG. 13 is a flowchart showing processing performed in the third embodiment.

FIG. 14 is a diagram showing mass attenuation coefficients of gold and iodine.

### DETAILED DESCRIPTION

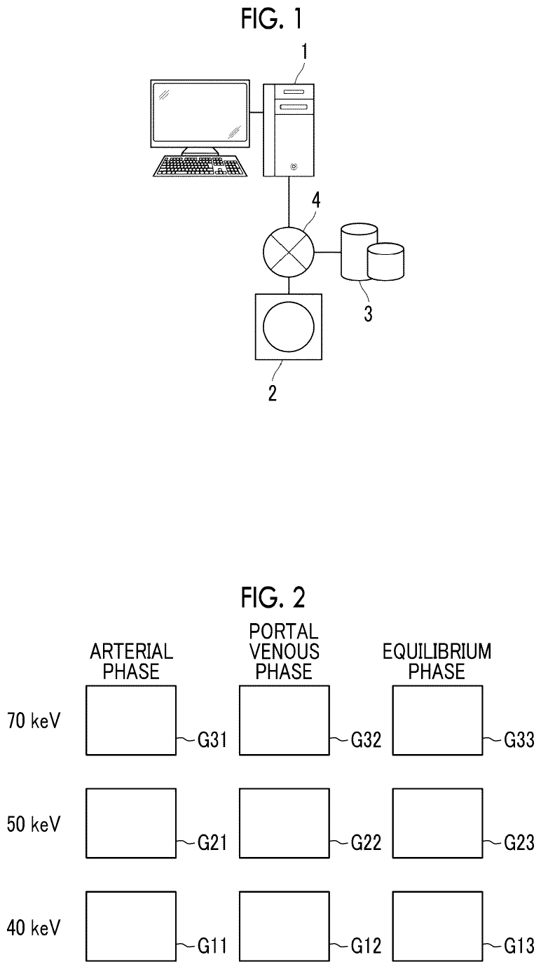

Hereinafter, a description of embodiments of the present disclosure will be made with reference to the accompanying drawings. First, a configuration of a medical information system to which an information processing apparatus according to a first embodiment according to the present disclosure is applied will be described. FIG. 1 is a diagram showing a schematic configuration of the medical information system. In the medical information system shown in FIG. 1, a computer 1 encompassing an information processing apparatus according to the first embodiment, a CT device 2, and an image storage server 3 are connected via a network 4 in a communicable state.

The computer 1 includes the information processing apparatus according to the first embodiment, and an information processing program of the first embodiment is installed in the computer 1. The computer 1 may be a workstation or a personal computer directly operated by a doctor who makes a diagnosis, or may be a server computer connected to the workstation or the personal computer via the network.

The CT device 2 is, for example, a dual-energy-type X-ray CT device (DECT device), and can acquire a virtual monochromatic X-ray image which is a CT image such as the image captured with any single energy by reconstructing first projection data and second projection data captured with two types of tube voltages for the same subject with various weightings.

The image storage server 3 is a computer that stores and manages various data, and comprises a large-capacity external storage device and database management software. The image storage server 3 communicates with another device via the wired or wireless network 4, and transmits and receives image data and the like to and from the other device. Specifically, the image storage server 3 acquires various data including the image data of the CT image generated by the CT device 2 via the network, and stores and manages the various data in the recording medium, such as the large-capacity external storage device. It should be noted that a storage format of the image data and the communication between the devices via the network 4 are based on a protocol such as digital imaging and communication in medicine (DICOM).

In the present embodiment, the CT device 2 generates a dynamic contrast CT image. For this reason, in the CT device 2, the combination of the first projection data and the second projection data having different energy levels is acquired in a plurality of contrast phases by imaging the subject at a plurality of timings while injecting a contrast agent into the liver of the subject. The contrast phase has three time phases of an arterial phase, a portal venous phase, and an equilibrium phase in accordance with an elapsed time from the start of the injection of the contrast agent. For example, the arterial phase is a time phase 40 seconds after the start of the injection of the contrast agent, the portal venous phase is a time phase 70 seconds after the start of the injection of the contrast agent, and the equilibrium phase is a time phase 180 seconds after the start of the injection of the contrast agent. The arterial phase, the portal venous phase, and the equilibrium phase are examples of a plurality of timings in the present disclosure.

In addition, in the present embodiment, the CT device 2 generates virtual monochromatic X-ray images having three types of energy levels by reconstructing the first projection data and the second projection data at three types of energy levels in each of the plurality of contrast phases. In the present embodiment, for example, virtual monochromatic X-ray images having energy levels of 40 keV, 50 keV, and 70 keV are generated in each of the arterial phase, the portal venous phase, and the equilibrium phase. 40 keV, 50 keV, and 70 keV are examples of predetermined energy levels in the present disclosure.

As a result, as shown in FIG. 2, virtual monochromatic X-ray images G11, G21, and G31 in the arterial phase, virtual monochromatic X-ray images G12, G22, and G32 in the portal venous phase, and virtual monochromatic X-ray images G13, G23, and G33 in the equilibrium phase are generated at each of the energy levels of 40 keV, 50 keV, and 70 keV. The generated virtual monochromatic X-ray images G11 to G13, G21 to G23, and G31 to G33 are transmitted to the image storage server 3 and stored.

In the dynamic contrast CT image, the appearance of a lesion included in an organ of a subject changes depending on the contrast phase. For example, in a case of malignant hepatocellular carcinoma, the density of the lesion is the highest in the arterial phase as compared with the surrounding tissue, and the density of the lesion is lower in the portal venous phase and the equilibrium phase in this order as compared with the surrounding tissue.

In addition, the appearance of the lesion also changes in accordance with the energy level in a case where the virtual monochromatic X-ray image is reconstructed. For example, even in the same contrast phase, the density of the lesion may be higher or lower than the density of the surrounding tissue in accordance with the energy level to be reconstructed. The way in which the change in the density occurs also varies depending on whether the lesion is malignant or benign.

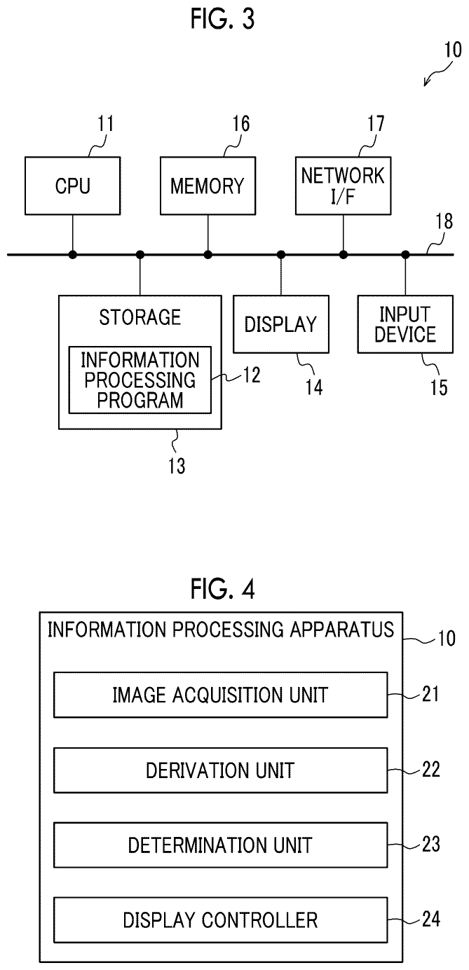

Next, the information processing apparatus according to the first embodiment will be described. FIG. 3 is a diagram showing a hardware configuration of the information processing apparatus according to the present embodiment. As shown in FIG. 3, an information processing apparatus 10 includes a central processing unit (CPU) 11, a non-volatile storage 13, and a memory 16 as a temporary storage area. Moreover, the information processing apparatus 10 includes a display 14, such as a liquid crystal display, an input device 15, such as a keyboard and a mouse, and a network interface (I/F) 17 connected to the network 4. The CPU 11, the storage 13, the display 14, the input device 15, the memory 16, and the network I/F 17 are connected to a bus 18. Note that the CPU 11 is an example of a processor according to the present disclosure.

The storage 13 is realized by a hard disk drive (HDD), a solid state drive (SSD), a flash memory, and the like. An information processing program 12 is stored in the storage 13 as the storage medium. The CPU 11 reads out the information processing program 12 from the storage 13, loads the information processing program 12 into the memory 16, and executes the loaded information processing program 12.

Next, a functional configuration of the information processing apparatus according to the first embodiment will be described. FIG. 4 is a diagram showing the functional configuration of the information processing apparatus according to the first embodiment. As shown in FIG. 4, the information processing apparatus 10 comprises an image acquisition unit 21, a derivation unit 22, a determination unit 23, and a display controller 24. By executing the information processing program 12 by the CPU 11, the CPU 11 functions as the image acquisition unit 21, the derivation unit 22, the determination unit 23, and the display controller 24.

The image acquisition unit 21 acquires the virtual monochromatic X-ray images G11 to G13, G21 to G23, and G31 to G33 to be processed from the image storage server 3 by an instruction from the input device 15 by the operator. In a case where the virtual monochromatic X-ray images G11 to G13, G21 to G23, and G31 to G33 are acquired from the image storage server 3 and stored in the storage 13, the image acquisition unit 21 acquires the virtual monochromatic X-ray images G11 to G13, G21 to G23, and G31 to G33 from the storage 13 for processing. The virtual monochromatic X-ray images G11 to G13, G21 to G23, and G31 to G33 are examples of a plurality of CT images of the present disclosure. In the following description, it is assumed that a plurality of virtual monochromatic X-ray images G11 to G13, G21 to G23, and G31 to G33 are represented by one reference numeral G0.

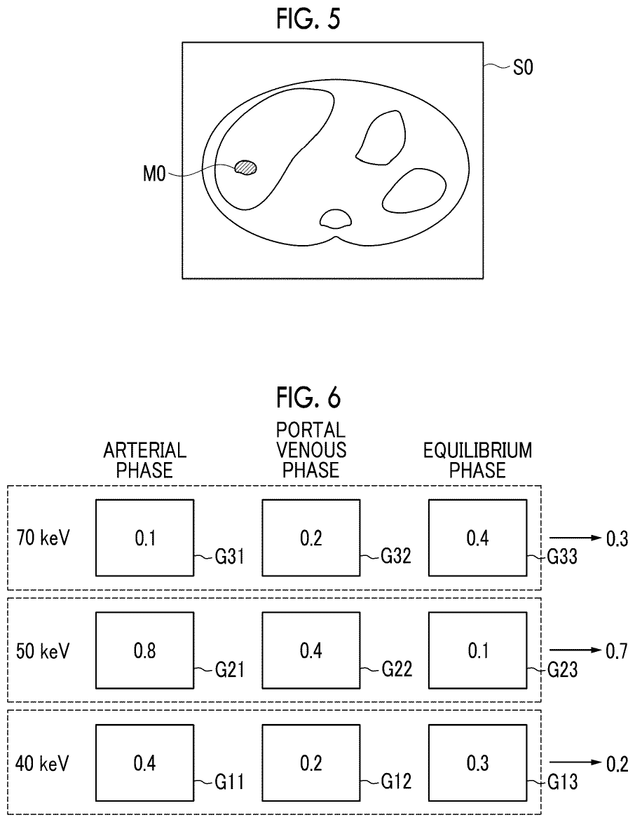

The derivation unit 22 derives visibility information related to the visibility of the target region included in each of the virtual monochromatic X-ray images G11 to G13, G21 to G23, and G31 to G33. For this purpose, the derivation unit 22 first extracts the lesion region as the target region from the virtual monochromatic X-ray images G0. FIG. 5 is a diagram for describing extraction of a lesion region. FIG. 5 shows a tomographic image S0 of one axial cross section of the virtual monochromatic X-ray images G0. For example, in a case where the virtual monochromatic X-ray images G0 are input, the derivation unit 22 extracts the lesion region M0 from the virtual monochromatic X-ray images G0 by using an extraction model that has been trained to extract a lesion of the liver.

The derivation unit 22 derives the feature value of the region including the lesion region M0 for each of the plurality of virtual monochromatic X-ray images G11 to G13, G21 to G23, and G31 to G33. In the present embodiment, the region including the lesion region M0 may be only the lesion region M0, may be a region of the lesion region M0 and a predetermined range around the lesion region M0, or may be a region of the lesion region M0 and the entire organ including the lesion region M0. In addition, as the feature value, for example, noise, contrast, edge, and the like of the region including the lesion region M0 can be used.

In a case where the feature value is noise, the derivation unit 22 derives noise of a region including the lesion region M0 for each of the plurality of virtual monochromatic X-ray images G11 to G13, G21 to G23, and G31 to G33. The noise can be obtained using the variance of the CT value. Here, the smaller the noise, the better the visibility of the lesion region M0.

In a case where the feature value is contrast, the derivation unit 22 derives contrast of a region including the lesion region M0 for each of the plurality of virtual monochromatic X-ray images G11 to G13, G21 to G23, and G31 to G33. Specifically, a difference in density between the lesion region M0 and the peripheral region thereof or a difference in density between the lesion region M0 and the region of the entire organ including the lesion region M0 is derived as the contrast. The larger the contrast, the better the visibility of the lesion region M0.

In a case where the feature value is the edge, the derivation unit 22 derives the strength of the edge of the lesion region M0 for each of the plurality of virtual monochromatic X-ray images G11 to G13, G21 to G23, and G31 to G33. The strength of the edge is a magnitude of a differential value at a boundary between the lesion region M0 and the peripheral region. As the strength of the edge is larger, the visibility of the lesion region M0 is improved.

The derivation unit 22 derives, as the visibility information, the amount of change in visibility of the lesion region M0 over time at each of the plurality of energy levels. Specifically, the amount of change in the feature value of the arterial phase, the portal venous phase, and the equilibrium phase is derived at each of the plurality of energy levels, and the maximum amount of change is derived as the visibility information. For example, as shown in FIG. 6, it is assumed that the contrast of the arterial phase, the portal venous phase, and the equilibrium phase at an energy level of 70 keV is 0.1, 0.2, and 0.4, respectively. In this case, the amount of change at 70 keV, that is, the visibility information is 0.3. In addition, in a case where the contrast of the arterial phase, the contrast of the portal venous phase, and the contrast of the equilibrium phase at the energy level of 50 keV are 0.8, 0.4, and 0.1, respectively, the amount of change at 50 keV, that is, the visibility information is 0.7. In addition, in a case where the contrast of the arterial phase, the contrast of the portal venous phase, and the contrast of the equilibrium phase at the energy level of 40 keV are 0.4, 0.2, and 0.3, respectively, the amount of change at 40 keV, that is, the visibility information is 0.2.

In the first embodiment, in a case where three virtual monochromatic X-ray images G0 of the arterial phase, the portal venous phase, and the equilibrium phase are input, the derivation unit 22 may derive the amount of change, that is, the visibility information for each of the plurality of energy levels using a derived model that is constructed to output the amount of change in the visibility of the lesion region M0 over time. In addition, for example, an indicator such as a contrast-to-noise ratio (CNR) may be used to derive the amount of change, that is, the visibility information from the feature value for the lesion region M0 based on the rule.

The determination unit 23 determines an image interpretation energy level for interpreting the virtual monochromatic X-ray images G0 among the plurality of energy levels based on the visibility information for each of the plurality of energy levels derived by the derivation unit 22. Specifically, the energy level at which the visibility information, that is, the amount of change is maximum, is determined as the image interpretation energy level. In the present embodiment, as described above, the visibility information at the energy levels of 70 keV, 50 keV, and 40 keV is 0.2, 0.7, and 0.2, respectively. Therefore, the determination unit 23 determines the visibility information, that is, the energy level of 50 keV having the maximum amount of change, as the image interpretation energy level.

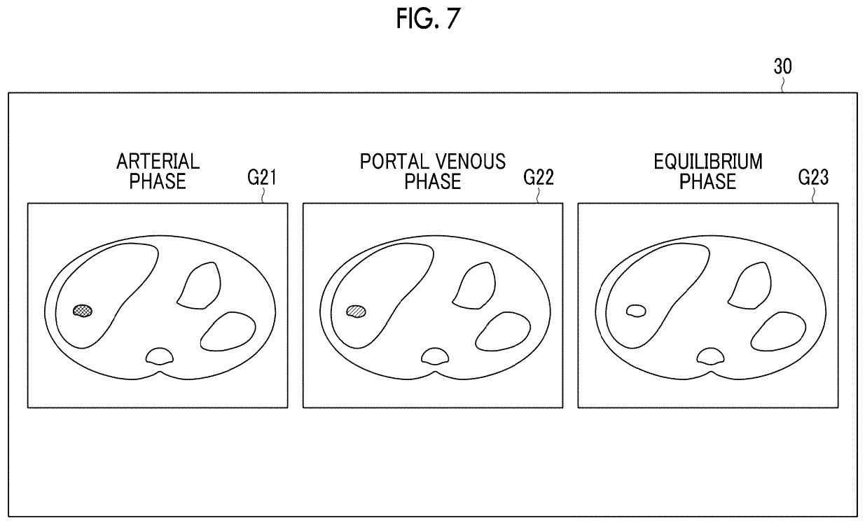

The display controller 24 sequentially displays the virtual monochromatic X-ray images G0 with the determined image interpretation energy levels in chronological order on the display 14. FIG. 7 is a diagram showing a display screen of the virtual monochromatic X-ray image. As shown in FIG. 7, on a display screen 30, the virtual monochromatic X-ray images G21 to G23 at the image interpretation energy level of 50 keV are displayed in order of the arterial phase, the portal venous phase, and the equilibrium phase from the left.

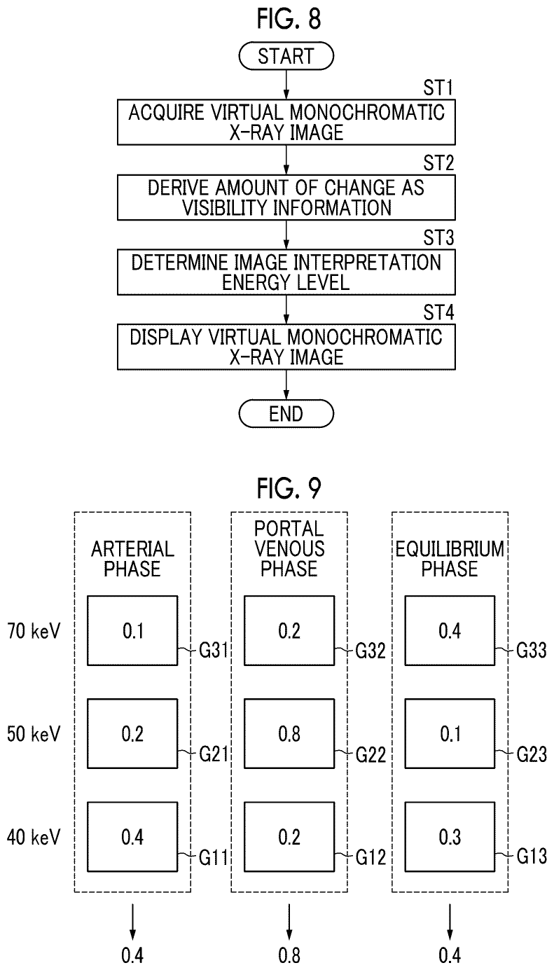

Next, the processing performed in the first embodiment will be described. FIG. 8 is a flowchart showing the processing performed in the first embodiment. First, the image acquisition unit 21 acquires the virtual monochromatic X-ray images G0 to be processed (step ST1). Then, the derivation unit 22 derives, as the visibility information, an amount of change representing a change in visibility of the lesion region M0 over time at each of the plurality of energy levels (step ST2). The determination unit 23 determines the image interpretation energy level by comparing the visibility information for each of the plurality of energy levels (step ST3). The display controller 24 sequentially displays the virtual monochromatic X-ray images G0 with the determined image interpretation energy levels in chronological order on the display 14 (step ST4), and the processing ends.

As described above, in the first embodiment, the amount of change representing the change in visibility of the lesion region M0 over time is derived as the visibility information, and the image interpretation energy level is determined by comparing the visibility information. Specifically, the energy level at which the amount of change is maximum is determined as the image interpretation energy level. Therefore, in the displayed virtual monochromatic X-ray image, the change of the lesion over time is more clearly shown than in the virtual monochromatic X-ray images G0 at other energy levels. Therefore, according to the present embodiment, in the virtual monochromatic X-ray image acquired by the dynamic contrast examination, it is possible to easily see the change of the lesion over time.

Next, a second embodiment of the present disclosure will be described. In the second embodiment, the functional configuration of the information processing apparatus is the same as the functional configuration of the information processing apparatus in the first embodiment, and only the processing performed by the derivation unit 22 and the determination unit 23 is different. Therefore, a detailed description of the configuration will not be shown here.

In the second embodiment, the derivation unit 22 derives a representative value representing the visibility of the lesion region M0 as the visibility information at each of the plurality of timings. Specifically, the derivation unit 22 first derives the feature value of the region including the lesion region M0 for each of the plurality of virtual monochromatic X-ray images G11 to G13, G21 to G23, and G31 to G33, in the same manner as in the above-described first embodiment. Then, in each of the arterial phase, the portal venous phase, and the equilibrium phase, a representative value representing the feature value of the lesion region M0 for each of the plurality of energy levels is derived as the visibility information.

For example, as shown in FIG. 9, it is assumed that the contrast at energy levels of 70 keV, 50 keV, and 40 keV in the arterial phase is 0.1, 0.2, and 0.4, respectively. In this case, the contrast is the highest at an energy level of 40 keV. Therefore, the derivation unit 22 derives 0.4, which is the feature value of the energy level of 40 keV for the arterial phase, as a representative value, that is, visibility information.

In addition, it is assumed that the contrast at energy levels of 70 keV, 50 keV, and 40 keV in the portal venous phase is 0.2, 0.8, and 0.2, respectively. In this case, the contrast is the highest at an energy level of 50 keV. Therefore, the derivation unit 22 derives 0.8, which is the feature value of the energy level of 50 keV for the portal venous phase, as a representative value, that is, visibility information.

In addition, it is assumed that the contrast at energy levels of 70 keV, 50 keV, and 40 keV in the equilibrium phase is 0.4, 0.1, and 0.3, respectively. In this case, the contrast is the highest at an energy level of 70 keV. Therefore, the derivation unit 22 derives 0.4, which is the feature value of the energy level of 70 keV for the equilibrium phase, as a representative value, that is, visibility information.

In the second embodiment, in a case where three virtual monochromatic X-ray images G0 of the arterial phase, the portal venous phase, and the equilibrium phase are input, the derivation unit 22 may derive a representative value representing the visibility of each of the arterial phase, the portal venous phase, and the equilibrium phase by using a derived model constructed to output a representative value representing the visibility of each of the arterial phase, the portal venous phase, and the equilibrium phase. In addition, for example, an indicator such as a contrast-to-noise ratio (CNR) may be used to derive the representative value from the feature value for the lesion region M0 based on the rule.

The determination unit 23 determines the image interpretation energy level for interpreting the virtual monochromatic X-ray images G0 among the plurality of energy levels, based on the visibility information for each of the arterial phase, the portal venous phase, and the equilibrium phase derived by the derivation unit 22. Specifically, the energy level at which the visibility information, that is, the representative value is optimal is determined as the image interpretation energy level. It should be noted that the representative value that is optimal varies in accordance with the type of the feature value. For example, in a case where the feature value is a contrast or an edge, the representative value at which the visibility information is optimal is the maximum representative value. On the other hand, in a case where the feature value is noise, the representative value at which the visibility information is optimal is the minimum representative value.

As described above, in a case where the contrast is the feature value, the representative value of the arterial phase is 0.4, the representative value of the portal venous phase is 0.8, and the representative value of the equilibrium phase is 0.4. Therefore, the determination unit 23 determines the visibility information, that is, the energy level of 50 keV having the maximum representative value, as the image interpretation energy level.

In the second embodiment, the display controller 24 sequentially displays the virtual monochromatic X-ray images G0 having the determined image interpretation energy level of 50 keV on the display 14 in chronological order. The display screen in the second embodiment is the same as the display screen in the first embodiment shown in FIG. 5, and thus, the detailed description thereof will not be shown here.

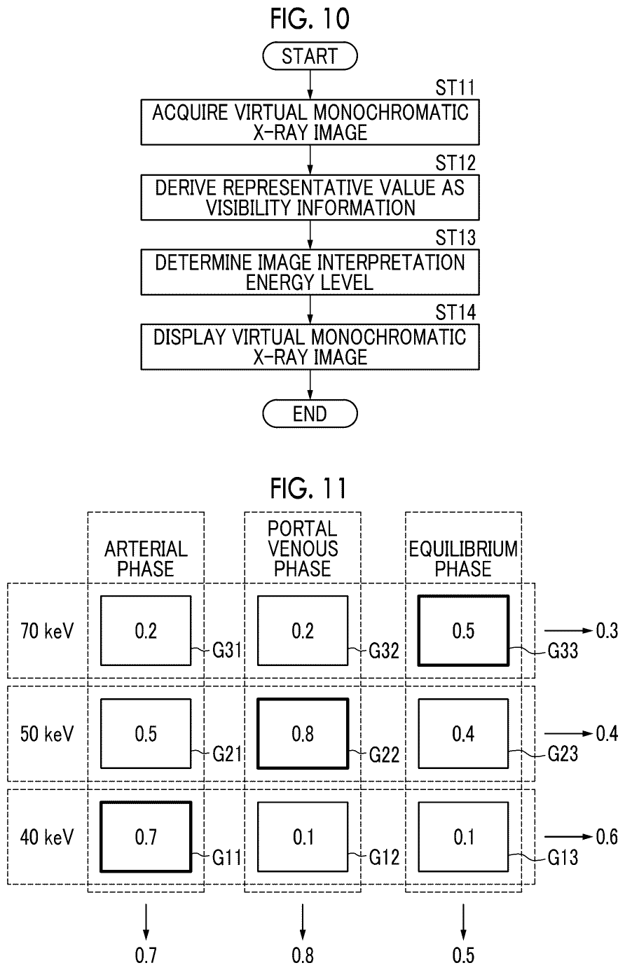

Next, the processing performed in the second embodiment will be described. FIG. 10 is a flowchart showing the processing performed in the second embodiment. First, the image acquisition unit 21 acquires the virtual monochromatic X-ray images G0 to be processed (step ST11). Then, the derivation unit 22 derives, as the visibility information, a representative value representing the feature value of the lesion region M0 for each of the plurality of energy levels in each of the arterial phase, the portal venous phase, and the equilibrium phase (step ST12). The determination unit 23 determines the image interpretation energy level by comparing the visibility information of the arterial phase, the portal venous phase, and the equilibrium phase (step ST13). The display controller 24 sequentially displays the virtual monochromatic X-ray images G0 with the determined image interpretation energy levels in chronological order on the display 14 (step ST14), and the processing ends.

As described above, in the second embodiment, the representative value representing the visibility of the lesion region M0 is derived as the visibility information at each of the plurality of timings, and the visibility information is compared to determine the image interpretation energy level. Specifically, the energy level at which the representative value is optimal is determined as the image interpretation energy level. Therefore, in the displayed virtual monochromatic X-ray image, the change of the lesion over time is more clearly shown than in the virtual monochromatic X-ray images G0 at other energy levels. Therefore, in the virtual monochromatic X-ray image acquired by the dynamic contrast examination, it is possible to easily see the change of the lesion over time.

Next, a description regarding a third embodiment of the present disclosure will be made. In the third embodiment, the functional configuration of the information processing apparatus is the same as the functional configuration of the information processing apparatus in the first embodiment, and only the processing performed by the derivation unit 22, the determination unit 23, and the display controller 24 is different. Therefore, a detailed description of the configuration will not be shown here.

In the third embodiment, the derivation unit 22 derives the feature value of the region including the lesion region M0 for each of the plurality of virtual monochromatic X-ray images G11 to G13, G21 to G23, and G31 to G33, in the same manner as in the first embodiment. Then, the derivation unit 22 derives the amount of change in visibility of the lesion region M0 over time in each of the plurality of energy levels, as in the first embodiment. In addition, as in the above-described second embodiment, the derivation unit 22 derives a representative value representing the feature value of the lesion region M0 for each of the plurality of energy levels in each of the arterial phase, the portal venous phase, and the equilibrium phase. In the third embodiment, the amount of change and the representative value are the visibility information.

FIG. 11 is a diagram for describing visibility information in the third embodiment. As shown in FIG. 11, it is assumed that the feature values of the arterial phase, the portal venous phase, and the equilibrium phase are 0.2, 0.2, and 0.5, respectively, at an energy level of 70 keV, the feature values of the arterial phase, the portal venous phase, and the equilibrium phase are 0.5, 0.8, and 0.4, respectively, at an energy level of 50 keV, and the feature values of the arterial phase, the portal venous phase, and the equilibrium phase are 0.7, 0.1, and 0.1, respectively, at an energy level of 40 keV. In this case, the amount of change derived at the energy level of 70 keV is 0.3, the amount of change derived at the energy level of 50 keV is 0.4, and the amount of change derived at the energy level of 40 keV is 0.6.

On the other hand, the representative value of the arterial phase is 0.7 at an energy level of 40 keV, the representative value of the portal venous phase is 0.8 at an energy level of 50 keV, and the representative value of the equilibrium phase is 0.5 at an energy level of 70 keV. In FIG. 11, the virtual monochromatic X-ray images G11, G22, and G33 that are representative values are surrounded by thick lines.

The determination unit 23 determines the image interpretation energy level based on the amount of change and the representative value included in the visibility information derived by the derivation unit 22. In the third embodiment, in a case where the image interpretation energy level is determined based on the amount of change as in the first embodiment, weighting is performed on the representative value for the amount of change in each of the plurality of energy levels, and the image interpretation energy level is determined in accordance with the weighted amount of change.

Here, in a case where the image interpretation energy level is determined only based on the amount of change, the energy level of 40 keV in which the amount of change is 0.6 is determined as the image interpretation energy level. On the other hand, in a case where the image interpretation energy level is determined only based on the representative values of the arterial phase, the portal venous phase, and the equilibrium phase, the energy level of 50 keV in which the representative value is 0.8 is determined as the image interpretation energy level. In the third embodiment, as the representative value is larger, the weighting for the amount of change in each of the plurality of energy levels is increased. For example, in a case where the representative value is 0.95 to 1.0, 0.3 is added as the weighting, in a case where the representative value is 0.85 to 0.95, 0.2 is added as the weighting, and in a case where the representative value is 0.7 to 0.85, 0.1 is added as the weighting, to the amount of change in the energy level of the representative value.

In the third embodiment, since the representative value is 0.8 at an energy level of 50 keV, 0.1 is added to 0.4 which is the amount of change at the energy level of 50 keV, so that the energy level of 50 keV is 0.5. In the present embodiment, in a case where the amount of change for each of the plurality of energy levels after weighting is compared, 0.6, which is the amount of change in the energy level of 40 keV, is still the maximum. Therefore, the determination unit 23 determines the energy level of 40 keV as the image interpretation energy level.

On the other hand, for example, in a case where the representative value of the energy level of 50 keV is 0.98, 0.3 is added to the amount of change in the energy level of 50 keV, so that the amount of change is 0.7. In this case, the amount of change in the energy level of 50 keV is larger than 0.6, which is the amount of change in the energy level of 40 keV. In this case, the determination unit 23 determines the energy level of 50 keV as the image interpretation energy level.

As in the first embodiment, the display controller 24 sequentially displays the virtual monochromatic X-ray images G0 with the determined image interpretation energy levels in chronological order on the display 14. Further, in the third embodiment, the image of the lesion region M0 included in the virtual monochromatic X-ray images G0 of the energy level from which the representative value is derived in each of the arterial phase, the portal venous phase, and the equilibrium phase is displayed in addition to the virtual monochromatic X-ray images G0 of the determined image interpretation energy level.

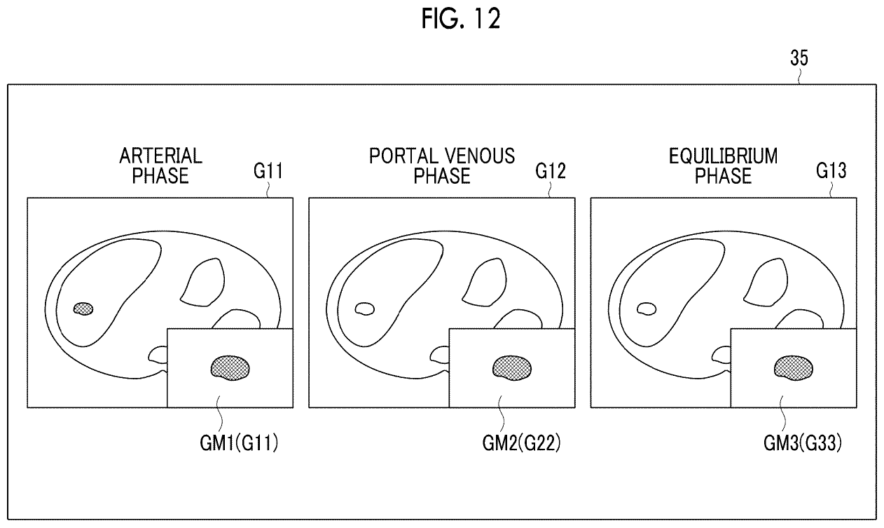

FIG. 12 is a diagram showing a display screen of a virtual monochromatic X-ray image in the third embodiment. As shown in FIG. 12, on a display screen 35, the virtual monochromatic X-ray images G11 to G13 at the image interpretation energy level of 40 keV are displayed in order of the arterial phase, the portal venous phase, and the equilibrium phase from the left. Further, in the virtual monochromatic X-ray image G11 of the arterial phase, an image of the lesion region M0 (referred to as a lesion region image GM1) included in the virtual monochromatic X-ray image G11 having an energy level of 40 keV at which the representative value is the maximum in the arterial phase is superimposed and displayed. In addition, in the virtual monochromatic X-ray image G12 of the portal venous phase, the lesion region image GM2 of the virtual monochromatic X-ray image G22 having an energy level of 50 keV, which is the maximum representative value in the portal venous phase, is superimposed and displayed. In addition, in the virtual monochromatic X-ray image G13 of the equilibrium phase, the lesion region image GM3 of the virtual monochromatic X-ray image G33 at the energy level of 70 keV, at which the representative value is the maximum in the equilibrium phase, is superimposed and displayed.

The operator may be able to switch between display and non-display of the lesion region images GM1 to GM3 by giving an instruction from the input device 15.

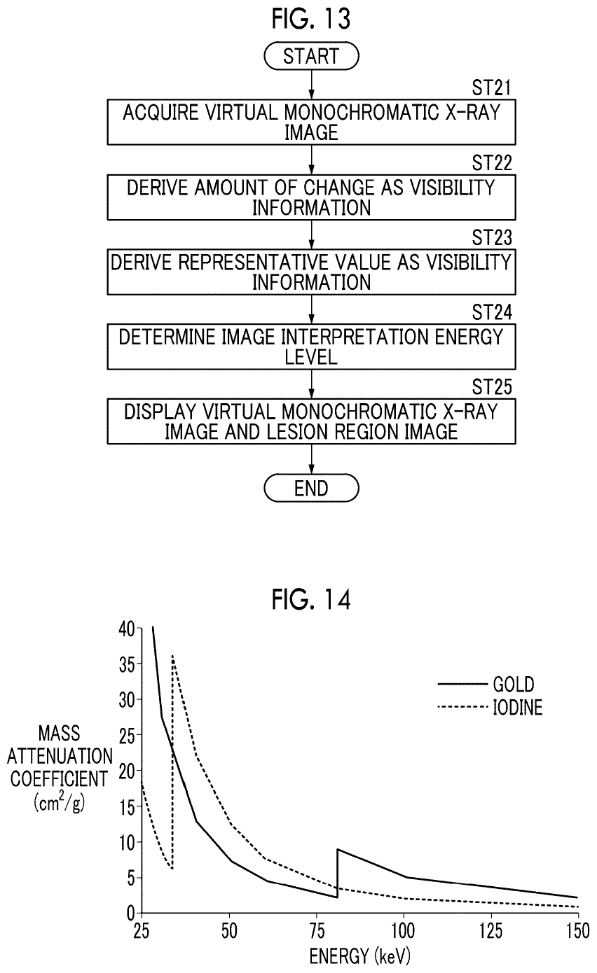

Next, the processing performed in the third embodiment will be described. FIG. 13 is a flowchart showing the processing performed in the third embodiment. First, the image acquisition unit 21 acquires the virtual monochromatic X-ray images G0 to be processed (step ST21). Then, the derivation unit 22 derives, as the visibility information, an amount of change representing a change in visibility of the lesion region M0 over time at each of the plurality of energy levels (step ST22). In addition, the derivation unit 22 derives, as the visibility information, a representative value representing the feature value of the lesion region M0 for each of the plurality of energy levels in each of the arterial phase, the portal venous phase, and the equilibrium phase (step ST23). The processing of step ST22 and the processing of step ST23 may be performed in reverse order or may be performed at the same time.

Next, the determination unit 23 determines the image interpretation energy level based on the amount of change and the representative value included in the visibility information derived by the derivation unit 22 (step ST24). The display controller 24 sequentially displays the virtual monochromatic X-ray images G0 at the determined image interpretation energy level together with the lesion region image in chronological order on the display 14 (step ST25), and the processing ends.

As described above, in the third embodiment, the image interpretation energy level is determined based on the amount of change representing the change in the visibility of the lesion region M0 over time and the representative value representing the visibility of the lesion region M0 at each of the plurality of timings. Therefore, it is possible to determine the image interpretation energy level at which the change of the lesion over time appears more clearly than the virtual monochromatic X-ray images G0 at other energy levels while considering the energy level at which the representative value is optimal.

In addition, in the third embodiment, in a case where the virtual monochromatic X-ray images G0 at the image interpretation energy level are displayed in chronological order, the lesion region image of the virtual monochromatic X-ray images G0 having the energy level at which the representative value is the maximum in each of the arterial phase, the portal venous phase, and the equilibrium phase is displayed. Therefore, it is possible to confirm the state of the lesion enhanced with the contrast agent in each of the contrast phases while confirming the change of the lesion over time.

There are various types of contrast agents such as iodine, iron, and gold. The energy level at which the contrast effect is high varies depending on the type of the contrast agent. FIG. 14 is a diagram showing mass attenuation coefficients of gold and iodine used as contrast agents. As shown in FIG. 14, since gold has a K absorption edge in the vicinity of 80 keV, the contrast effect is high at an energy level in the vicinity of 80 keV. On the other hand, iodine has a K absorption edge in the vicinity of 30 keV, and thus has a high contrast effect at an energy level in the vicinity of 30 keV.

In addition, the time during which the contrast agent remains in the body varies in accordance with the type of the contrast agent. Further, the ease of uptake of the contrast agent varies depending on the type of cell. For example, there may be a lesion in which a large amount of iodine is taken in first and a large amount of gold is taken in thereafter.

Therefore, in the examination using the contrast agent, a plurality of types of contrast agents may be mixed and used. For example, in a case where a contrast agent obtained by mixing gold and iodine is used, in a case where the virtual monochromatic X-ray images G0 are generated at energy levels of 30 keV and 80 keV, and the energy level of 30 keV is determined as the image interpretation energy level, it is possible to confirm a change in degree of enhancement of a lesion due to iodine over time. In addition, in a case where the energy level of 80 keV is determined as the image interpretation energy level, a change in the degree of enhancement of the lesion due to gold over time can be confirmed. In this case, which energy level is determined as the image interpretation energy level may be determined in accordance with the visibility information, that is, the magnitude of the amount of change or the magnitude of the representative value derived from the virtual monochromatic X-ray images G0 in the above-described first to third embodiments.

In each of the above-described embodiments, the CT device 2 is a dual-energy-type X-ray CT device (DECT device), but the present invention is not limited thereto. The CT device 2 may be a photon counting CT device (PCCT device). In the PCCT device, it is possible to acquire a plurality of pieces of projection data having different energy levels acquired for each energy bin, and it is possible to derive the virtual monochromatic X-ray images G0 by using and reconstructing the plurality of pieces of projection data at any single energy level.

On the other hand, it is possible to derive the material decomposition image from a plurality of pieces of projection data having different energy levels acquired by the DECT device and the PCCT device. The material decomposition image is a CT image obtained by a material decomposition technology of decomposing a substance included in a subject using projection data corresponding to a plurality of energy levels by using the fact that the absorption characteristics of radiation differ for each substance.

Hereinafter, in a case where the CT device 2 is the PCCT device, the derivation of the material decomposition image in a case where the projection data at three energy levels is acquired by the PCCT device will be described. In a case where linear attenuation coefficients of a fat, a muscle, and a contrast agent (iodine) included in the subject under examination using the contrast agent at a certain energy level E are μ1, μ2, and μ3, respectively, the projection data P at the energy level E can be represented by the following Expression (1).

$$P ( E ) = - μ 1 ( E ) \times 1 - μ2 ( E ) \times 2 - μ3 ( E ) \times 3 \qquad ( 1 )$$

x1, x2, and x3 are densities of fat, muscle, and a contrast agent. The material decomposition is to obtain x1, x2, and x3 in Formula (1).

Here, in the PCCT device, the above-described Expression (1) at three energy levels can be obtained by using the acquired projection data of three energies, and thus the unknown variables x1, x2, and x3 can be obtained.

In the present embodiment, for example, in a case where iodine and gold are used by being mixed together, a material decomposition image of iodine and a material decomposition image of gold are generated by using projection data at a plurality of energy levels acquired by the PCCT device. Since iodine and gold have different energy levels with high contrast effects, the iodine material decomposition image and the gold material decomposition image are examples of a plurality of CT images of the present disclosure.

Even in a case where the material decomposition images of iodine and gold are generated in this way, the image interpretation energy level may be determined in the above-described first to third embodiments. In this case, the energy level with a high contrast effect of iodine or the energy level with a high contrast effect of gold is determined as the image interpretation energy level.

In each of the embodiments described above, various processors shown below can be used as the hardware structure of the processing units that execute various types of processing, such as the image acquisition unit 21, the derivation unit 22, the determination unit 23, and the display controller 24. As described above, the above-described various processors include a programmable logic device (PLD) as a processor of which the circuit configuration can be changed after manufacture, such as a field-programmable gate array (FPGA), a dedicated electrical circuit as a processor having a dedicated circuit configuration for executing specific processing such as an application-specific integrated circuit (ASIC), and the like, in addition to the CPU as a general-purpose processor that functions as various processing units by executing software (program).

One processing unit may be configured by one of the various processors, or may be configured by a combination of the same or different types of two or more processors (for example, a combination of a plurality of FPGAs or a combination of the CPU and the FPGA). Further, a plurality of processing units may be configured with one processor.

As an example where a plurality of processing units are configured by one processor, first, there is a form in which one processor is configured by a combination of one or more CPUs and software as typified by a computer, such as a client or a server, and this processor functions as a plurality of processing units. Second, as represented by a system-on-chip (SoC) or the like, there is a form of using a processor for realizing the function of the entire system including a plurality of processing units with one integrated circuit (IC) chip. As described above, the various types of processing units are configured using one or more of the above-described various types of processors as a hardware structure.

Furthermore, as the hardware structure of the various processors, more specifically, an electrical circuit (circuitry) in which circuit elements such as semiconductor elements are combined can be used.

Hereinafter, the appendices of the present disclosure will be described.

### APPENDIX 1

An information processing apparatus comprising:
a processor,
in which the processor is configured to:
   acquire a plurality of CT images derived by reconstructing a plurality of pieces of projection data having different energy levels, which are acquired by imaging a subject at a plurality of timings, at a plurality of predetermined energy levels;
   derive visibility information related to visibility of a target region included in each of the plurality of CT images; and
   determine an image interpretation energy level for interpreting the CT image among the plurality of energy levels based on the visibility information.

### APPENDIX 2

The information processing apparatus according to appendix 1,
in which the processor is configured to:
   derive an amount of change representing a change in the visibility of the target region over time at each of the plurality of energy levels, as the visibility information; and
   determine the image interpretation energy level by comparing the visibility information for each of the plurality of energy levels.

### APPENDIX 3

The information processing apparatus according to appendix 2,
in which the processor is configured to determine an energy level at which the amount of change is maximum among the plurality of energy levels, as the image interpretation energy level.

### APPENDIX 4

The information processing apparatus according to appendix 1, in which the processor is configured to:
derive a representative value representing the visibility of the target region at each of the plurality of timings, as the visibility information; and
determine the image interpretation energy level by comparing the representative value for each of the plurality of timings.

### APPENDIX 5

The information processing apparatus according to appendix 4,
in which the processor is configured to determine the image interpretation energy level based on an energy level at which the representative value at each of the plurality of timings is optimal.

### APPENDIX 6

The information processing apparatus according to appendix 1,
in which the processor is configured to:
   derive an amount of change in the visibility of the target region over time at each of the plurality of energy levels and a representative value representing the visibility of the target region at each of the plurality of timings, as the visibility information; and
   determine the image interpretation energy level based on the amount of change and the representative value.

### APPENDIX 7

The information processing apparatus according to appendix 6,
in which the processor is configured to:
   weight at least one of the amount of change or the representative value in accordance with the amount of change and the representative value; and
   determine the image interpretation energy level based on the weighted amount of change and representative value.

### APPENDIX 8

The information processing apparatus according to any one of appendices 1 to 7,
in which the processor is configured to sequentially display the plurality of CT images at the image interpretation energy level in chronological order.

### APPENDIX 9

The information processing apparatus according to appendix 8,
in which the processor is configured to further display an image of the target region included in the CT image having a highest visibility in association with the plurality of CT images at the image interpretation energy levels at each of the plurality of timings.

### APPENDIX 10

The information processing apparatus according to appendix 9,
in which the processor is configured to switch between display and non-display of the image of the target region in accordance with an instruction from an operator.

### APPENDIX 11

The information processing apparatus according to any one of appendices 1 to 10,
in which the CT image is a virtual monochromatic X-ray image derived by reconstructing the plurality of pieces of projection data having different energy levels at the plurality of predetermined energy levels.

### APPENDIX 12

The information processing apparatus according to any one of appendices 1 to 10,
in which the CT image is a material decomposition image derived by reconstructing the plurality of pieces of projection data having different energy levels at the plurality of predetermined energy levels.

### APPENDIX 13

The information processing apparatus according to any one of appendices 1 to 12,
in which the processor is configured to derive the visibility information based on a feature value of a region including the target region included in the CT image.

### APPENDIX 14

The information processing apparatus according to any one of appendices 1 to 13,
in which the plurality of timings are based on a contrast phase in a case where a contrast agent is injected into the subject.

### APPENDIX 15

The information processing apparatus according to any one of appendices 1 to 14,
in which the target region is a lesion region.

### APPENDIX 16

An information processing method comprising:
causing a computer to
   acquire a plurality of CT images derived by reconstructing a plurality of pieces of projection data having different energy levels, which are acquired by imaging a subject at a plurality of timings, at a plurality of predetermined energy levels,
   derive visibility information related to visibility of a target region included in each of the plurality of CT images, and
   determine an image interpretation energy level for interpreting the CT image among the plurality of energy levels based on the visibility information.

### APPENDIX 17

An information processing program causing a computer to execute:
a procedure of acquiring a plurality of CT images derived by reconstructing a plurality of pieces of projection data having different energy levels, which are acquired by imaging a subject at a plurality of timings, at a plurality of predetermined energy levels;
a procedure of deriving visibility information related to visibility of a target region included in each of the plurality of CT images; and
a procedure of determining an image interpretation energy level for interpreting the CT image among the plurality of energy levels based on the visibility information.

What is claimed is:

1. An information processing apparatus comprising:
a processor,
wherein the processor is configured to:
acquire a plurality of CT images derived by reconstructing a plurality of pieces of projection data having different energy levels, which are acquired by imaging a subject at a plurality of timings, at a plurality of predetermined energy levels;
derive visibility information related to visibility of a target region included in each of the plurality of CT images; and
determine an image interpretation energy level for interpreting the CT image among the plurality of energy levels based on the visibility information.

2. The information processing apparatus according to claim 1,
wherein the processor is configured to:
derive an amount of change representing a change in the visibility of the target region over time at each of the plurality of energy levels, as the visibility information; and
determine the image interpretation energy level by comparing the visibility information for each of the plurality of energy levels.

3. The information processing apparatus according to claim 2,
wherein the processor is configured to determine an energy level at which the amount of change is maximum among the plurality of energy levels, as the image interpretation energy level.

4. The information processing apparatus according to claim 1,
wherein the processor is configured to:
derive a representative value representing the visibility of the target region at each of the plurality of timings, as the visibility information; and
determine the image interpretation energy level by comparing the representative value for each of the plurality of timings.

5. The information processing apparatus according to claim 4,
wherein the processor is configured to determine the image interpretation energy level based on an energy level at which the representative value at each of the plurality of timings is optimal.

6. The information processing apparatus according to claim 1,
wherein the processor is configured to:
derive an amount of change in the visibility of the target region over time at each of the plurality of energy levels and a representative value representing the visibility of the target region at each of the plurality of timings, as the visibility information; and
determine the image interpretation energy level based on the amount of change and the representative value.

7. The information processing apparatus according to claim 6,
wherein the processor is configured to:
weight at least one of the amount of change or the representative value in accordance with the amount of change and the representative value; and
determine the image interpretation energy level based on the weighted amount of change and representative value.

8. The information processing apparatus according to claim 1,
wherein the processor is configured to sequentially display the plurality of CT images at the image interpretation energy level in chronological order.

9. The information processing apparatus according to claim 8,
wherein the processor is configured to further display an image of the target region included in the CT image having a highest visibility in association with the plurality of CT images at the image interpretation energy levels at each of the plurality of timings.

10. The information processing apparatus according to claim 9,
wherein the processor is configured to switch between display and non-display of the image of the target region in accordance with an instruction from an operator.

11. The information processing apparatus according to claim 1,
wherein the CT image is a virtual monochromatic X-ray image derived by reconstructing the plurality of pieces of projection data having different energy levels at the plurality of predetermined energy levels.

12. The information processing apparatus according to claim 1,
wherein the CT image is a material decomposition image derived by reconstructing the plurality of pieces of projection data having different energy levels at the plurality of predetermined energy levels.

13. The information processing apparatus according to claim 1,
wherein the processor is configured to derive the visibility information based on a feature value of a region including the target region included in the CT image.

14. The information processing apparatus according to claim 1,
wherein the plurality of timings are based on a contrast phase in a case where a contrast agent is injected into the subject.

15. The information processing apparatus according to claim 1,
wherein the target region is a lesion region.

16. An information processing method comprising:
causing a computer to
acquire a plurality of CT images derived by reconstructing a plurality of pieces of projection data having different energy levels, which are acquired by imaging a subject at a plurality of timings, at a plurality of predetermined energy levels,
derive visibility information related to visibility of a target region included in each of the plurality of CT images, and
determine an image interpretation energy level for interpreting the CT image among the plurality of energy levels based on the visibility information.

17. A non-transitory computer-readable storage medium that stores an information processing program causing a computer to execute:
a procedure of acquiring a plurality of CT images derived by reconstructing a plurality of pieces of projection data having different energy levels, which are acquired by imaging a subject at a plurality of timings, at a plurality of predetermined energy levels;
a procedure of deriving visibility information related to visibility of a target region included in each of the plurality of CT images; and
a procedure of determining an image interpretation energy level for interpreting the CT image among the plurality of energy levels based on the visibility information.
